(19) United States
(12) Patent Application Publication
(10) Pub. No.: US 2018/0146925 A1
(43) Pub. Date: May 31, 2018

(54) Tentacular Electrode Catheter Apparatus

(71) Applicant: Jamil MOGUL, Saratoga, CA (US)

(72) Inventor: Jamil MOGUL, Saratoga, CA (US)

(21) Appl. No.: 14/982,043

(22) Filed: Dec. 29, 2015

Related U.S. Application Data
(60) Provisional application No. 62/097,551, filed on Dec. 29, 2014.

(51) Int. Cl. A61B 5/00; A61B 5/0452; A61B 5/042; A61B 18/14

(52) U.S. Cl. CPC A61B 5/6859; A61B 5/0452; A61B 2018/00357; A61B 18/1492; A61B 5/4836; A61B 5/0422

(57) ABSTRACT

A tentacular electrode catheter having a proximal shaft component and a steerable distal shaft component with a polar array of multiple bi-directionally deflectable tentacles extending longitudinally around the distal shaft component. The distal end of each tentacle has a tip electrode for recording bio-potentials, pacing, and delivering and depositing RF energy. The tentacles are advantageously attached to the proximal shaft component at its junction with the distal shaft component and extend over a portion of the distal shaft component. The portion of the distal shaft component that is axially at the center of the polar array of tentacles thus acts as a centralizer and stabilizer for the tentacles, and is accordingly referred to as a centralizer/stabilizer distal shaft component.

### FIELD OF THE INVENTION

The present invention relates generally to catheter apparatus and more particularly to an improved electrode catheter for use in diagnosing cardiac arrhythmias and delivering radiofrequency (RF) energy to problematic endocardial tissue

### BACKGROUND OF THE INVENTION

An electrophysiology study (EP test or EP study) is a minimally invasive procedure that tests the electrical conduction system of the heart to assess the electrical activity and conduction pathways of the heart. During EPS, sinus rhythm as well as supraventricular and ventricular arrhythmias of baseline cardiac intervals is recorded. The study is indicated to investigate the cause, location of origin, and best treatment for various abnormal heart rhythms. This type of study is performed by an electrophysiologist and using a single or multiple catheters situated within the heart through a vein or artery.

Cardiac arrhythmia, as a disease, occurs when the heart rate is persistently out of the normal range. There are various cardiac arrhythmias; including supraventricular tachycardia, atrial flutter, atrial fibrillation, Wolf-Parkinson-White syndrome, accelerated idioventricular rhythm, ventricular tachycardia, Torsades de Pointes (polymorphic ventricular tachycardia), ventricular fibrillation and asystol. These diseases are treated by drugs or devices called electrode catheters which deliver radiofrequency (RF) energy to a problematic endocardial tissue in order to ablate or denature it. The electrode catheters are also used for diagnosis of cardiac arrhythmias.

Atrial fibrillation (AF) occurs due to various factors. An underlying atrial substrate plays a role in intra-atrial conduction delay. In addition, atrial ectopic beats initiate AF. An ectopic focus is an excitable group of cells that causes a premature heart beat outside the normally functioning sinoatrial node of the human heart. Acute occurrence is usually non-life-threatening, but chronic occurrence can progress into tachycardia, bradycardia or ventricular fibrillation. It has been found that the major sources of these ectopic beats appear to be the pulmonary veins, although other endocardial sources are found to be also contributory factors. Many studies suggest that AF may be treated by RF ablation within or around the pulmonary veins. Various reports have identified pulmonary vein ectopic foci as AF sources and described the abolition of these sources. Stopping pulmonary foci from firing and thereby identifying the culprit veins has led to a clinical technique for achieving electrical isolation of all four of the pulmonary veins in an effort to abolish the initiating triggers of AF. The recent results suggest that around 70% of patients with paroxysmal AF can have a long-term normal heart rate by means of this technique.

The pulmonary veins along with the bronchial veins are part of the venous drainage system of the lungs. The pulmonary veins are large blood vessels that receive oxygenated blood from the lungs and drain it into the left atrium of the heart. There are four pulmonary veins, two from each lung. The pulmonary veins are among the few veins that carry oxygenated blood. The pulmonary veins drain oxygenated blood to the left atrium. Nearly 70% of people have four pulmonary veins, two on the left and the other two on the right.

Some people have pulmonary veins on the right or left side merging to a common ostium before entering the left atrium. Other people have branches of the pulmonary veins that can open into the left atrium through separate orifices. The pulmonary veins are covered by a 1 to 3 centimeter long myocardial layer or sleeve of atrial tissue. A majority of the vein is composed of circular fibers, but there are longitudinal fibers that may constitute the electrical paths through which pulmonary vein foci, deep within the vein, are able to excite the atrial myocardium.

The variable anatomy of the pulmonary vein can cause difficulties in performing the pulmonary vein isolation via RF ablation. Therefore, understanding the individual patient's anatomy is critical to the success of pulmonary vein isolation. For this purpose, prior to performing pulmonary vein isolation via RF ablation, a magnetic resonance imaging scan or trans-esophageal echocardiogram is needed in order to achieve accurate elucidation of the pulmonary vein anatomy.

Normally, in a pulmonary vein isolation procedure, a patient is prepped and draped using sterile surgical techniques. Access is obtained through the femoral veins for multi-electrode EP catheters. An EP catheter is inserted into the vein and placed in the coronary sinus for recording and pacing. Other EP catheters are placed in the right atrium and the His bundle for recording and pacing. Intracardiac echo-cardiography is used as a standard practice to provide visualization of key anatomic structures during the procedure.

Using the intracardiac echocardiography as a visualization aid, an atrial trans-septal puncture is made for accessing the left atrium. A mapping catheter with its distal portion containing 10 to 20 electrodes is inserted through the puncture, placed at the left atrium. Then the distal portion of the catheter is deflected to orthogonally form a circular shape which is placed within the antrum or ostium of one of the pulmonary veins to record the bio-potentials (bio-electric signals) of the ostium. The circular shaped catheter is then maneuvered to access other pulmonary veins and their ostium bio-electric signals are recorded.

Once the pulmonary vein bio-potentials are observed and the related ectopic foci are identified, an RF ablation catheter is inserted through the same or another trans-septal puncture and placed at one of the foci. Then, RF energy is delivered and deposited at the foci, and the catheter is placed at all other foci in order to abolish them via RF ablation, or in other words, to isolate each pulmonary vein.

The disappearance of pulmonary vein bio-potentials can be more easily assessed while observing the dissociated automaticity within the isolated pulmonary vein tissue. Alternatively, pacing from each electrode of the mapping catheter to the pulmonary vein tissue can be performed to help confirm exit conduction block from or electrical isolation of the pulmonary vein if dissociated activity is not observed.

The Problem Addressed

The shape of the orthogonal circular distal portion of the mapping catheter used in the pulmonary isolation procedure is formed as an offset, closed loop orthogonally emanating from the proximal shaft (main body) of the catheter. This makes the loop cantilevered (supported on one side of the loop); and as a result, the loop becomes unstable during heart beats, making or maintaining a less than optimal contact with the pulmonary vein ostium. Also, the loop cannot be centered or aligned accurately around the ostium of a pulmonary vein with full assurance.

Additionally, one using a mapping catheter sometimes has difficulty in straightening the loop while inside the left atrium of a heart in an effort to withdraw the catheter. Moreover, in some cases another catheter is trans-septally used for RF ablation so as to isolate the pulmonary veins. This widens the trans-septal puncture or requires another trans-septal puncture, increasing the likelihood of infection and/or injury risks, and lengthening puncture closing and wound healing time.

### Solution

To address the aforementioned issues, a tentacular electrode catheter has been designed having a steerable, and possibly rotatable, distal shaft component with a polar array of multiple, bi-directionally deflectable tentacles placed longitudinally around a portion thereof and having at least one electrode disposed at the distal end of each tentacle.

### BRIEF SUMMARY OF THE INVENTION

Briefly, a presently preferred embodiment of the present invention includes a catheter having a handle, a proximal shaft, and a steerable, and possibly rotatable, distal shaft. Attached to the distal end of the proximal shaft is a polar array of multiple, bi-directionally deflectable tentacles placed longitudinally around and extending over a proximal portion of the distal shaft. The distal end of each tentacle has a tip electrode for recording bio-potentials, pacing and delivering and depositing RF energy. The portion of the distal shaft component that is axially at the center of the polar array of tentacles acts as a centralizer and stabilizer of the array, and is thus sometimes referred to herein as the centralizer/stabilizer distal shaft.

To locate the tentacular catheter up/down, or sideways, by rotating the catheter, the distal shaft component can be steered to access any pulmonary vein ostium. Once a pulmonary vein ostium is accessed, the distal shaft component is inserted and advanced into the pulmonary vein until the tentacles are near the ostium. The tentacles are then deflected to a required size of the ostium, and the distal shaft component is further advanced until the tip electrodes of the deflected tentacles reach the desired position whereupon diagnosis and/or ablation can be conducted.

The distal shaft component also has its own tip electrode and ring electrodes, and thus can also be used with un-deflected tentacles as a standard catheter.

A principal advantage of the present invention is that it provides an improved electrode catheter having electrodes that can be centered, or aligned, accurately around the ostium of a pulmonary vein with assurance of positioning accuracy.

Another advantage of the present invention is that it provides optimal contact with the pulmonary vein ostium under diagnosis and/or treatment.

Still another advantage of the present invention is that it provides an electrode catheter having means for facilitating ease of entry and withdrawal from an intra-cardiac site to be analyzed and/or treated.

Yet another advantage of the present invention is that it provides an electrode catheter which has a dual purpose of electrophysiology diagnosis and therapy via RF ablation eliminating a need for a second electrode catheter for RF ablation whereby increasing procedural efficiency and providing an optimal diagnostic/therapeutic approach.

A corollary to the above advantage is that the present invention eliminates a need for another trans-septal puncture or widening of the existing trans-septal puncture whereby decreasing the post-procedure healing time of the trans-septal puncture (reducing hospital stay—saving costs) and/or minimizing risks of injuries or complications.

These and other advantages of the present invention will become apparent to those skilled in the art after having read the following detailed disclosure of embodiments illustrated in the several figures of the drawing.

### BRIEF DESCRIPTION OF THE SEVERAL VIEWS IN THE DRAWING

FIG. 1 is a top plan view of an embodiment of the present invention;

FIG. 2A is an enlarged view of a portion of the embodiment of FIG. 1 showing details of the distal end of the catheter with the tentacle electrodes retracted and nested about the centralizer/stabilizer steerable distal shaft component;

FIG. 2B is an end view of the catheter detailed view of FIG. 2A showing the tentacle electrodes nested about the centralizer/stabilizer steerable distal shaft;

FIG. 3 is another plan view of the embodiment of FIGS. 1 and 2 with the tentacle electrodes deflected away from the centralizer/stabilizer steerable distal shaft;

FIG. 4A is an enlarged view of the portion of the embodiment depicted in FIG. 3 showing the tentacle electrodes deflected away from the centralizer/stabilizer steerable distal shaft;

FIG. 4B is an end view of the catheter detailed view of FIG. 4A showing the tentacle electrodes deflected away from the centralizer/stabilizer steerable distal shaft;

FIG. 5 is another plan view of the embodiment of FIG. 1 showing the steerable distal shaft component steered to the right of the handle in accordance with the present invention;

FIG. 6A is an enlarged view of the portion of the embodiment depicted in FIG. 5 showing details of the steerable distal shaft component deflected laterally;

FIG. 6B is an end view of the detailed view of FIG. 6A showing the steerable distal shaft component steered laterally;

FIG. 7 is an open view of the handle of the embodiment of FIG. 1 showing the internal components including the electrode wires, steering wires and lever, and tentacle deflection wires and knob;

FIG. 8 is an enlarged view of the front end of the handle showing convergence of the electrode wires, steering wires, and tentacle deflection wires as they pass into the end of the proximal shaft;

FIG. 9 is another enlarged view of the front end of the handle showing details of the connection of the steering wires to the steering lever;

FIG. 10 is an enlarged view of the rear end of the handle showing details of the exiting of the electrode wires from the handle and entrance into the signal carrying cable, and connection and operational detail of the tentacle deflection wires and knob;

FIG. 11 is a view showing a partially broken segment of the catheter and schematically illustrating the junction of the distal end of the proximal shaft tubing, the proximal end of the centralizer/stabilizer steerable distal shaft component and the proximal ends of the deflectable tentacles;

FIG. 12A is cut-away view showing details of the distal tip electrode, tip electrode wire, steering wire connection and flat spring, as well as a ring electrode and connection of a ring electrode wire thereto, of the centralizer/stabilizer steerable distal shaft;

FIG. 12B is a transverse cross-section of the steerable distal shaft component taken along the line B-B of FIG. 12A;

FIG. 13 is a partially broken segment showing the junction of the steerable shaft tubing and the less flexible tubing piece, and details of the steering wire and steering mechanism of the steerable distal shaft;

FIG. 14A is a partially broken view of the distal end of a deflectable tentacle showing various details thereof;

FIG. 14B is a transverse cross-section of the deflectable tentacle taken along the line B-B of FIG. 14A;

FIG. 15 is a plan view of an alternative embodiment of the present invention having a steerable distal shaft component with a non-steerable centralizer/stabilizer shaft and deflectable tentacles;

FIG. 16A is an enlarged view of a portion of the embodiment of FIG. 15 showing details of the distal end of the catheter with the tentacles retracted and nested about the non-steerable centralizer/stabilizer;

FIG. 16B is an end view of the detailed view of FIG. 15A showing the tentacles retracted and nested about the non-steerable centralizer/stabilizer;

FIG. 17 is another plan view of the alternative embodiment of FIG. 15 showing the tentacles deflected away from the non-steerable centralizer/stabilizer;

FIG. 18A is an enlarged view of the portion of the alternative embodiment depicted in FIG. 15 showing the tentacles deflected away from the centralizer/stabilizer;

FIG. 18B is an end view of the detailed view of FIG. 18A showing the tentacles deflected away from the non-steerable centralizer/stabilizer;

FIG. 19 is another plan view of the embodiment of FIG. 15 showing the steerable distal shaft component turned laterally in accordance with the present invention;

FIG. 20A is an enlarged view of the portion of the embodiment depicted in FIG. 15 showing details of the steerable distal shaft component turned laterally;

FIG. 20B is an end view of the detailed view of FIG. 20A showing the steerable distal shaft component turned laterally;

FIG. 21 is a partially broken view showing further details of the area referenced in FIG. 16A;

FIG. 21A is a transverse cross section taken along the line A-A in FIG. 21 and showing further details of the distribution of the tentacles, steering wires and distal shaft flat spring at the section line A-A;

FIG. 22 is a partially broken view showing further details of the area referenced in FIG. 15; and

FIGS. 22A and 22B show additional details of the connection of the inner support tube to the outer support tube shown in FIG. 22.

### DETAILED DESCRIPTION OF THE EMBODIMENTS

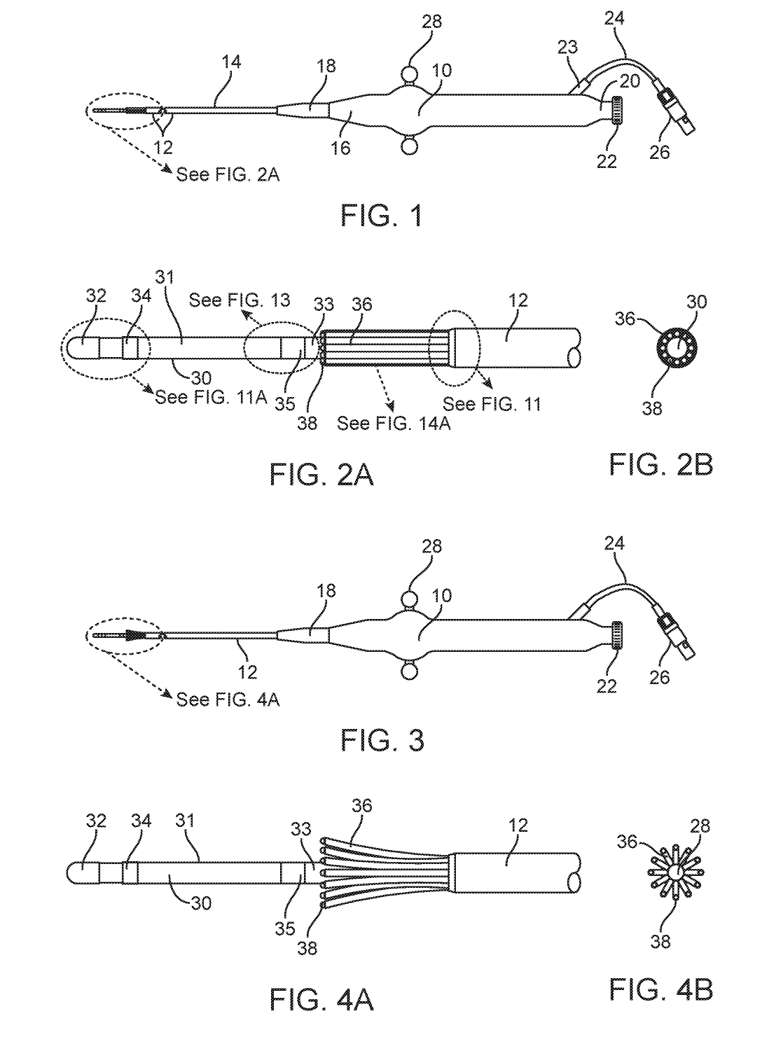

Referring now to FIG. 1 which is a top plan view of an embodiment of an improved electrode catheter in accordance with the present invention and includes a handle 10 having the proximal shaft 12 of an elongated catheter 14 affixed to its front end 16 via a strain relief fitting 18. Note by the illustrated break lines that the length of the proximal shaft is substantially longer than that depicted. Positioned at the rear or butt end 20 of handle 10 is a deflection control knob 22. Extending out of the handle near the end 20 via a strain relief fitting 23 is a data cable 24 having a connector 26 provided at its distal end. Extending out of longitudinally running slots (not shown) formed on each side of the handle 10 are the respective ends of a catheter steering lever 28.

As is shown more clearly in FIG. 2A, which is an enlarged view of a portion of the embodiment of FIG. 1 and shows certain details of the catheter 14, it will be seen that the distal end section of the catheter includes a tubular member forming a distal shaft component 30 including a flexible and steerable portion 31 having a distal tip electrode 32, a less flexible portion 33, a distal ring electrode 34, and a plurality (12 in this embodiment) of tentacles 36 shown in their retracted or un-deflected positions nested about the centralizer/stabilizer portion 33 of the steerable distal shaft component 30. Each of the deflectable tentacles includes a tip electrode 38.

Note that in this embodiment the distal shaft component tubing is fabricated in two parts; the flexible part 31 for accommodating the catheter steering function, and the relatively less flexible part 33 for providing a Centralizing/Stabilizing function for the deflectable tentacles 36. The adjacent ends of these two parts are bonded together with a tubing piece 35 as will be further explained below.

FIG. 2B is an end view of the catheter segment illustrated in FIG. 2A further showing the tentacles 36 and their tip electrodes 38 nested about the distal shaft component 30.

FIG. 3 is another plan view of the embodiment of FIG. 1 with the tentacles 36 shown deflected away from the distal shaft distal shaft component 30 and into a sensing/treatment position as will be explained below.

FIG. 4A is an enlarged view of the referenced portion of the embodiment depicted in FIG. 3 showing the tentacles 36 deflected away from the centralizing/stabilizing portion 33 of the distal shaft component 30 and into a sensing/treatment deployment as will be further explained below.

FIG. 4B is an end view of the depiction of FIG. 4A showing the tentacles deflected away from the shaft 30 as shown in FIG. 4A.

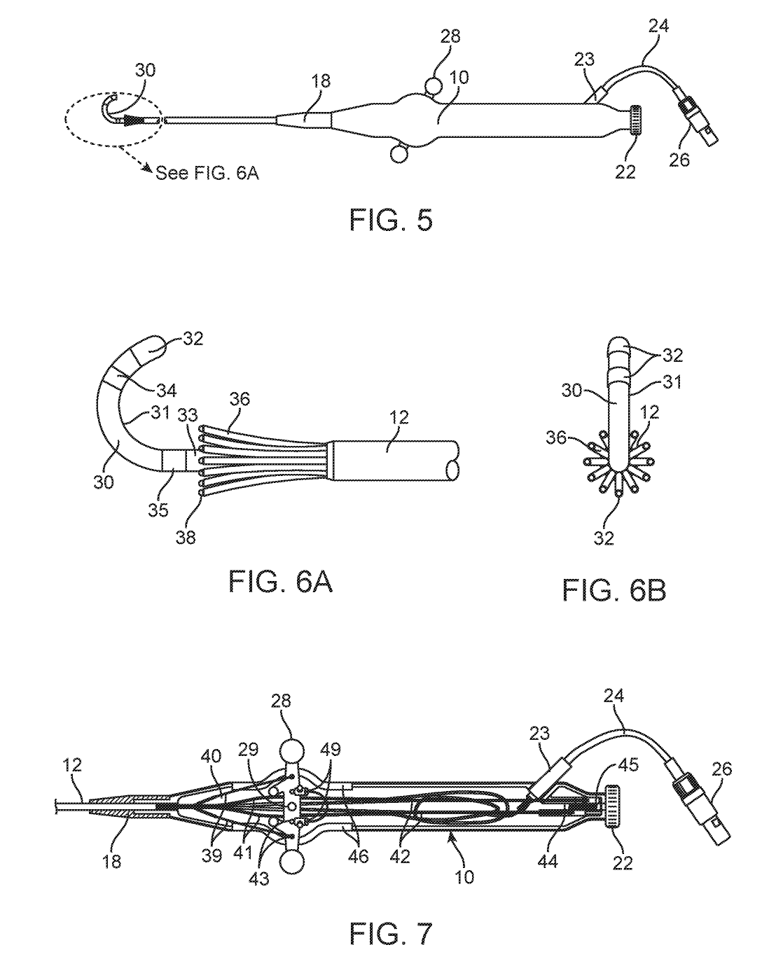

FIG. 5 is another plan view of the embodiment of FIG. 1 showing the flexible steerable distal shaft portion 31 deflected to the right side of the catheter (upwardly as depicted) in accordance with the present invention.

FIG. 6A is an enlarged view of the portion of the embodiment called out in FIG. 5 and more clearly showing the more flexible steerable part 31 of the distal shaft 30 turned laterally to the right (upwardly as shown in the drawing) and the tentacles 36 deflected outwardly and away from the less flexible centralizing/stabilizing part 33 of the distal shaft component 30.

FIG. 6B is an end view of the depiction in FIG. 6A showing the steerable distal shaft tubing part 31 turned relative to the axis of the proximal shaft 12, and the tentacles 36 deflected outwardly relative to the less flexible tubing part 33.

FIG. 7 is a plan view of the handle 10 of the embodiment of FIG. 1 with the upper handle housing part removed to show the internal components thereof including steering lever 28, steering cable 39, steering wire 40, electrode wires 41, tentacle deflection wires 42 and the wire attachment mechanism 44 associated with the deflection adjustment screw 45 attached to tentacle deflection control knob 22. Also shown are friction pads 46 lining the lever openings on each side of lever 28 to frictionally engage the upper and lower surfaces of the lever and hold it in place each time it is adjusted in position about a pivot pin 29 to redirect the steerable portion of the distal shaft.

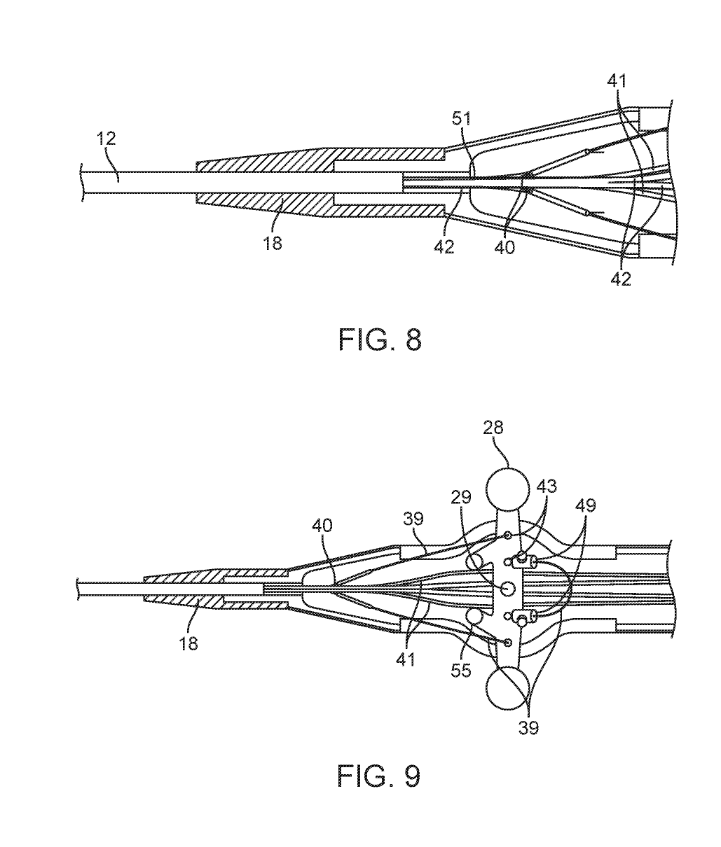

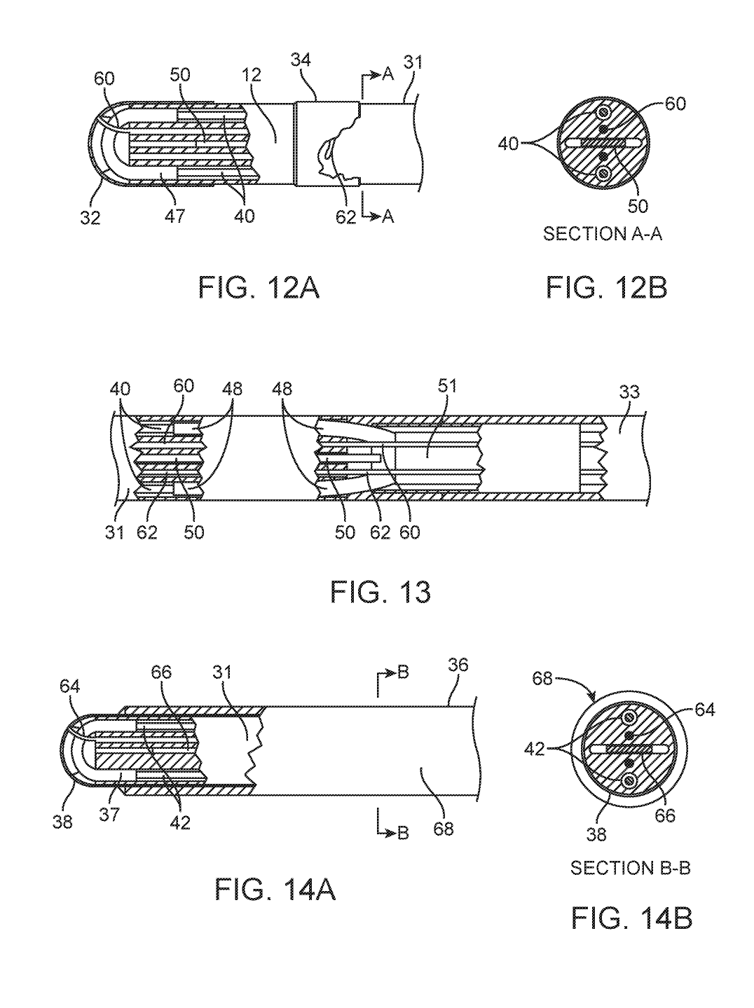

As will be understood from this figure and the further showings in FIGS. 8, 9 and 12A, the distal shaft steering function is preferably achieved by means of a relatively short flexible cable 39 extended through openings 43 on each side of the steering lever pivot 29, with each end thereof directed toward the near end of proximal shaft 12. The cable ends are then crimp-connected (see FIG. 8 for clarity) to the ends of a single length of steering wire 40 that is routed through guide tubes 51 and alignment tubes 48 (FIG. 13) to and through a steering wire U-bend tube 47 (FIG. 12A) embedded in the distal end of distal shaft component 30.

Note in FIGS. 12A,12B and 13 that the steerable portion 31 of distal shaft component 30 also has a flat spring 50 extending along the centerline thereof, the plane of such flat spring lying orthogonal to the plane of the U-bend tube 47 to control the bending direction of the shaft 12. Alternatively, a substitute for the flat spring 50 can be a U-shaped wire form (not shown) encompassing dimensionally the same space. Proper alignment of the steering lever 28 with the bending direction of the flexible part 31 (FIG. 6A) of the distal tube is secured by screw clamps 49 on cable 39.

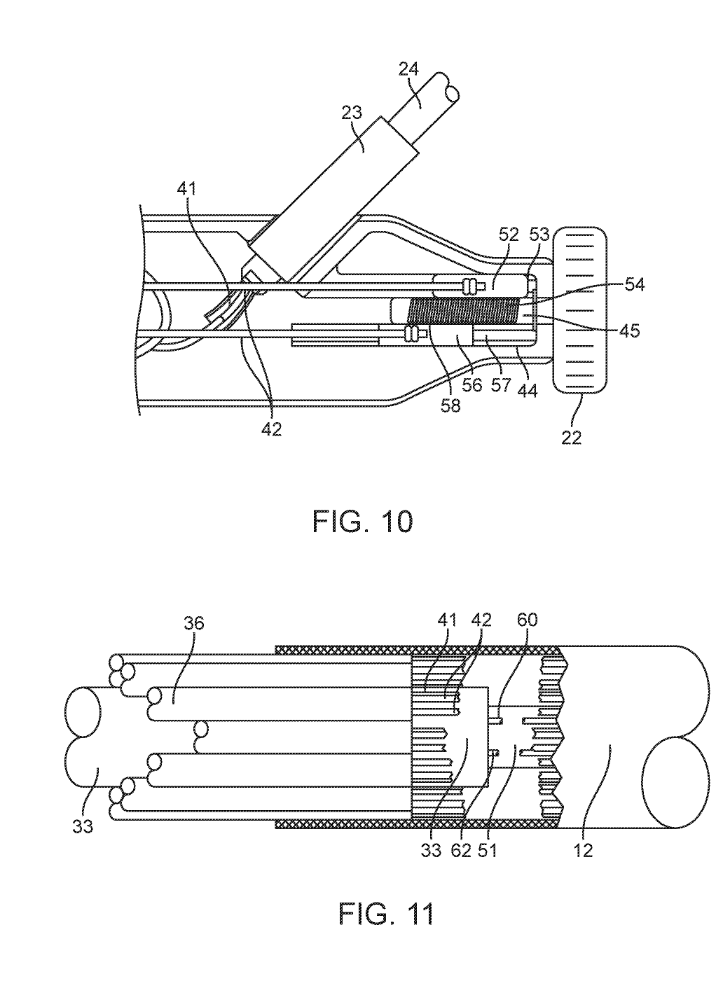

Similarly, deflection of the tentacles 36 is achieved by means of a plurality of deflection control wires 42 each of which has one end attached to a first slider 52 (see also FIG. 10—a metal strip with a thread profile protrusion 54) slideably captured in a first metal strip track 53 with its protrusion 54 engaging the threads on one side of the rotatable but fixed position screw 45 attached to knob 22, and extends through guide tube 51, proximal shaft 12, one of the deflectable tentacles 36, a U-bend tube 37 (FIG. 14A) embedded in the distal end of the tentacle, and then back through the tentacle, proximal shaft 12, guide tube 51, and handle 10 where it is attached to a second slider 56 slideably captured in a second track 57, the slider 56 having a protrusion 58 engaging an opposite side of the screw 45.

It will thus be understood that rotation of the knob 22 in one direction causes the tentacles 36 to simultaneously deflect away from the underlying tube portion 33, and rotation of the knob 22 in the opposite direction will cause the tentacles 36 to retract back towards the underlying tube portion 33.

FIG. 8 is an enlarged view of the front end of the handle 10 cut away to more clearly show convergence of the steering wires 40, electrode wires 41, and tentacle deflection wires 42 as they pass into the end of the guide tube 51 and proximal shaft 12.

FIG. 9 is another enlarged view of the front end of the handle 10 cut away to more clearly show details of the connection of the steering wire/cable 39 to the steering lever 28 which contains protrusions 55 for providing an extra tug (pull) to the steering wire when the steering lever 28 is actuated (rotated) to the limiting point). More pull of the steering wire aids in more steerability of the flexible steerable part 31 of the distal shaft 30.

FIG. 10 is an enlarged view of the rear end of the handle cut away to show details of the exiting of the electrode wires 41 from the handle 10 and entrance into the signal carrying cable 24, and connection and operational detail of the tentacle deflection wires 42 and knob 22.

FIG. 11 is a view showing a partially broken segment of the catheter and schematically illustrating the junction of the distal end of the proximal shaft tubing 12, the proximal end of the centralizer/stabilizer portion 33 of the distal shaft, and the proximal ends of the deflectable tentacles 36.

FIG. 12A is cut-away view showing further details of the distal tip electrode 32, steering wire U-bend tube 47 and flat spring 50, as well as a ring electrode 34 and connection of a ring electrode wire thereto, of the centralizer/stabilizer steerable distal shaft;

FIG. 12B is a transverse cross-section of the steerable distal shaft component 30 taken along the line A-A of FIG. 12A to show the positions of the steering wire 40, flat spring 50, as well as the connection wires for tip electrode 32 and ring electrode 34.

FIG. 13 is a partially broken segment showing the junction of the steerable shaft tubing 31 and the less flexible tubing piece 33 characterized above as the centralizer/stabilizer portion of the distal shaft component 30, and details of the steering wire guide tube 51, steering wire alignment tubes 48, and distal shaft flat spring 50.

FIG. 14A is a partially broken view of the distal end of a deflectable tentacle 36 showing various details thereof including the tentacle tip electrode 38, the tip electrode wire 64, the tentacle deflection wire U-bend tube 37, the tentacle deflection wire 42 and tentacle tubing flat spring 66. Note that the tentacle tubing 31 is disposed within a flexible tentacle sleeve 68 to ensure that when the tentacles 36 are in contact with a very small ostium of a pulmonary vein, the tip electrode 38 of each of the tentacles 36 is physically spaced (isolated) from the next tip electrode 38 in order to avoid any electrical shortage during recording/pacing or RF-energy deposition.

FIG. 14B is a transverse cross-section of the deflectable tentacle taken along the line B-B of FIG. 14A and showing the positioning within the tentacle of the components identified in FIG. 14A.

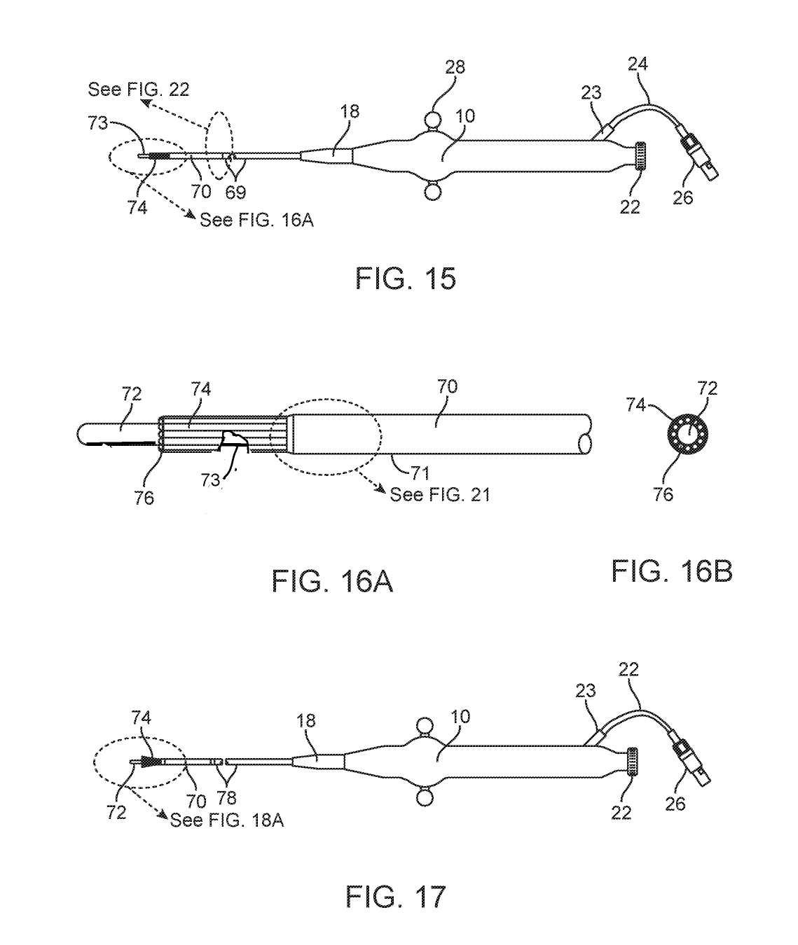

FIG. 15 is a plan view of an alternative embodiment of the present invention having a proximal shaft 69, a steerable distal shaft component 70 with a less flexible or non-flexible portion 71 (FIG. 71), and a non-steerable extension 72 forming a centralizer/stabilizer element affixed to and extending beyond the distal end of the steerable portion of component 70. In this alternative embodiment, the polar array of multiple, bi-directionally deflectable tentacles 74 is affixed to the distal end of the steerable portion 71 (FIG. 16A) of distal shaft component 70, with the tentacles 74 disposed around and longitudinally extending over a proximal portion 73 of the extension 72. As in the previously described embodiment, and as is more clearly shown in the referenced FIG. 16A, each tentacle 74 also includes a tentacle tip electrode 76.

FIG. 16B is an end view of the detailed view of FIG. 16A showing the tentacles 74 retracted and nested about the non-steerable centralizer/stabilizer shaft 72.

FIG. 17 is another plan view of the alternative embodiment of FIG. 15 showing the tentacles 74 deflected away from the proximal portion 73 of centralizer/stabilizer shaft extension 72.

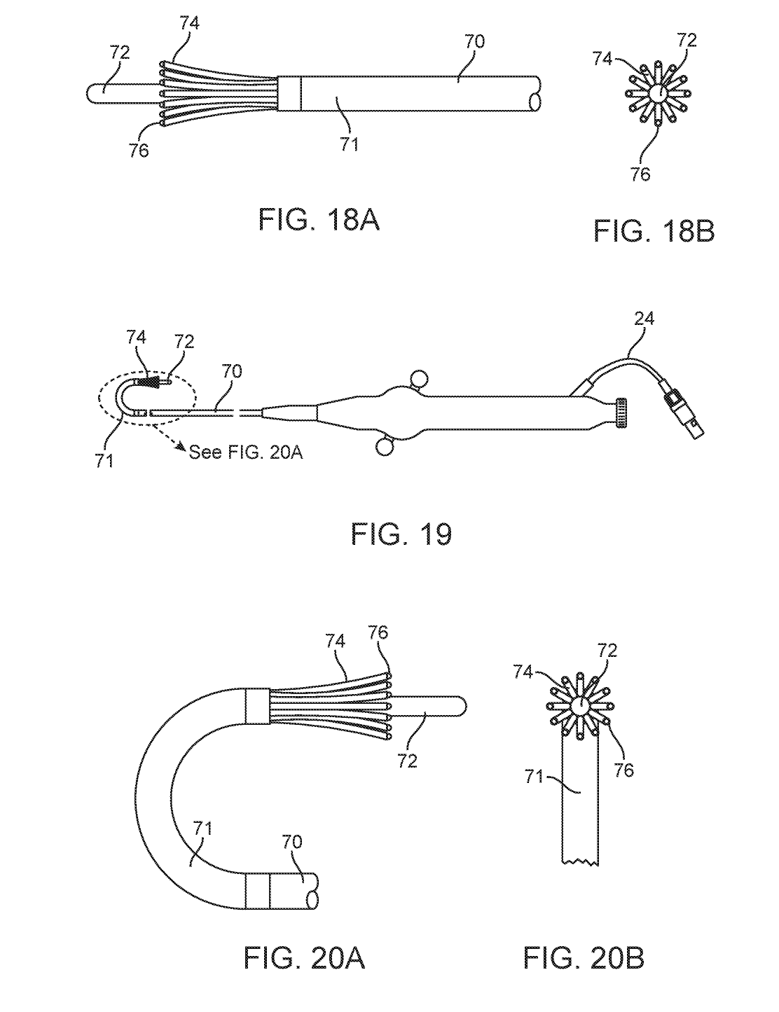

FIG. 18A is an enlarged plan view depicting the tentacles 74 deflected away from the centralizer/stabilizer shaft extension 72.

FIG. 18B is an end view of the showing in FIG. 18A depicting the tentacles 74 deflected away from the centralizer/stabilizer shaft extension 72.

FIG. 19 is another plan view of the embodiment of FIG. 15 showing the steerable distal shaft component 71 turned laterally (upwardly as depicted), and the tentacles 74 deflected (as is more clearly shown in FIG. 20A) in accordance with the present invention.

FIG. 20B is an end view of the detailed view of FIG. 20A showing the steerable distal shaft component 70 steered laterally.

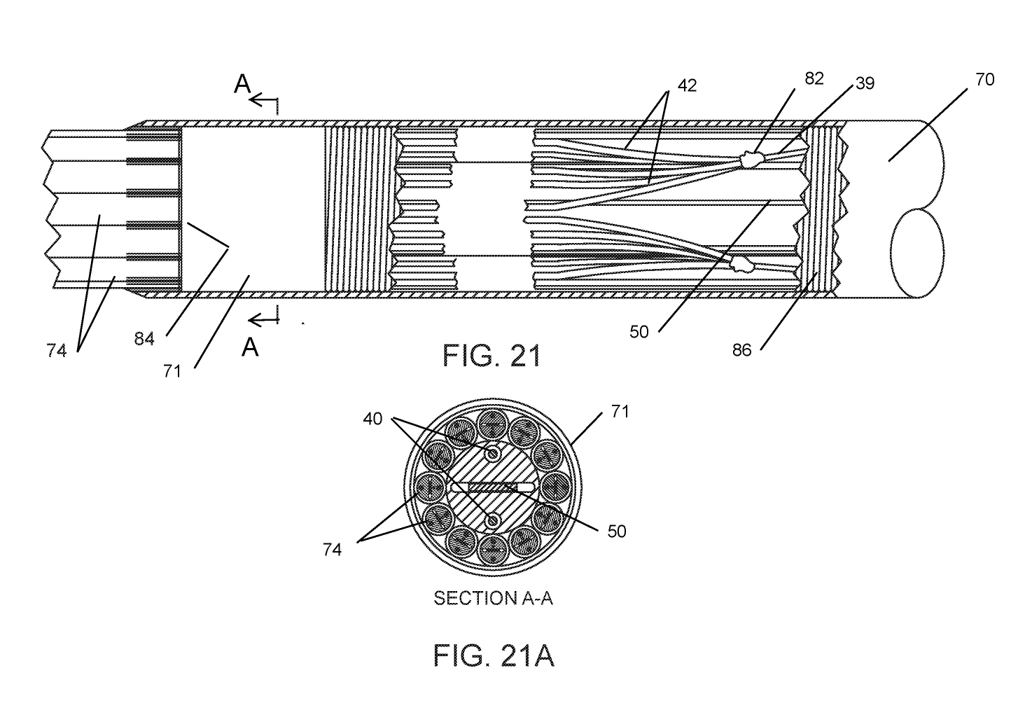

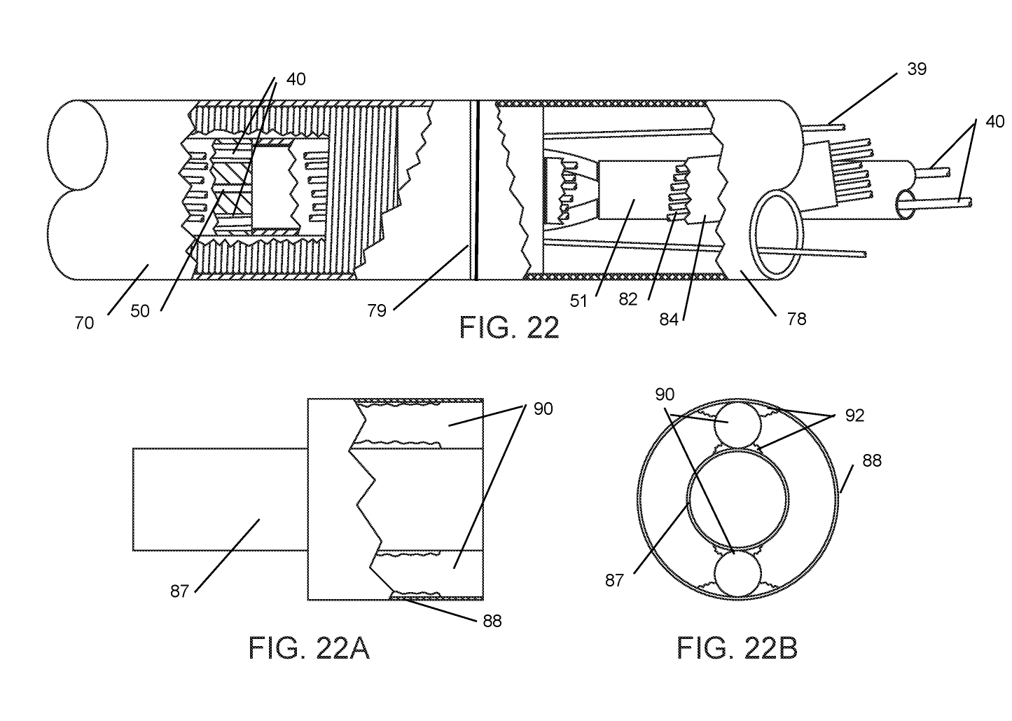

FIGS. 21-22B show further details of the above described embodiments. FIG. 21 is a partially broken view showing further details of the area referenced in FIG. 16A; more specifically, it shows the manner in which the tentacle deflection wires 42 are ganged together at 82 and attached (soldered) to the tentacle deflection puller wires 39, and the way in which the tentacles 74 are adhesively attached and bonded to the tentacle base tube 71 at 84 and the outer distal shaft tubing 70 in the embodiment of FIG. 15. Note that a zero-pitch coil 86 is used to accommodate and maintain the flexing integrity of the outer distal shaft 70.

FIG. 21A is a transverse cross section taken along the line A-A in FIG. 21 and showing further details of the distribution of the tentacles 74, steering wires 40 and distal shaft flat spring 50.

FIG. 22 is a partially broken view showing further details of the area referenced in FIG. 15; more specifically, the manner in which the steerable distal shaft 70 is attached to the distal end of the proximal shaft tubing 78 at 79, the arrangement of the steering wire guide tube 51, the steering wire alignment tubes 48, flat spring 50, the tentacle tip electrode wires 82, the tentacle tip electrode wire sleeve 84. and the inner support tube 86 and outer support tube 88.

FIGS. 22A and 22B show additional details of the connection of the inner support tube 87 to the outer support tube 88 using pins 90 and solder 92.

As is clearly shown in the several figures of the drawing, the steering function in each of the disclosed two embodiments of the catheter steering function is separate from the tentacle deflection function, and thus achieves the sought-for solutions to the prior art problems mentioned above.

Although the present invention has been described above in terms of specific embodiments, and various applications have been suggested, it is anticipated that after reading the foregoing disclosure, numerous other embodiments and applications of the present invention will become apparent to those skilled in the art. It is therefore intended that this disclosure be considered as exemplary rather than limiting, and that the following claims be interpreted as covering all alternatives, modifications and embodiments as fall within the true spirit and scope of the invention.

1. An electrode catheter for use in diagnosing cardiac arrhythmias and delivering radiofrequency (RF) energy to problematic endocardial tissue, comprising:
a handle;
a catheter shaft having a proximal shaft component with one end attached to the handle, and a second end, and a distal shaft component with a proximal end attached to the second end of the proximal shaft component, and a distal end, the distal shaft component having a steerable portion and a non-steerable portion;
a polar array of multiple, elongated, bi-directionally deflectable tentacles disposed around the shaft and having a proximal end thereof attached to the shaft, the tentacles each extending over at least a part of the non-steerable portion and in a generally longitudinal direction relative thereto, the distal end of each tentacle having an electrode affixed to its distal end;
first means associated with the handle and operatively connected to the steerable portion of the distal shaft component for enabling a user of the catheter to steer the distal end thereof in an orthogonal direction relative to the axial direction of the non-steerable portion of the distal shaft component; and
second means associated with the handle and operatively connected to the array of tentacles for enabling a user of the catheter to bi-directionally deflect the distal ends of the tentacles relative to the steerable portion of the catheter, whereby the tentacle electrodes can be operatively positioned for diagnosis and/or treatment.

2. An electrode catheter as recited in claim 1 wherein the non-steerable portion of the distal shaft component is attached to the second end of the proximal shaft component.

3. An electrode catheter as recited in claim 2 wherein the tentacles are attached to the second end of the proximal shaft component and extend over the non-steerable component of the distal shaft component.

4. An electrode catheter as recited in claim 3 wherein the non-steerable portion of the distal shaft component provides a centralizing and stabilizing function for the tentacles.

5. An electrode catheter as recited in claim 1 wherein a tip electrode is affixed to the distal end of the distal shaft component.

6. An electrode catheter as recited in claim 5 wherein at least one ring electrode is disposed along the length of the steerable portion of the distal shaft component.

7. An electrode catheter as recited in claim 1 wherein the first means includes a user-engageable steering lever affixed to the handle and connected to the distal end of the distal shaft component by wires extending along the length of the catheter shaft.

8. An electrode catheter as recited in claim 7 wherein the second means includes a user-engageable screw mechanism affixed to the handle and connected to the distal ends of the tentacles by wires extending along the length of the catheter shaft.

9. An electrode catheter as recited in claim 8 wherein the screw mechanism includes a threaded screw that engages first and second slides driven in opposite directions by the screw, and wires extending through the catheter shaft and tentacles to the distal ends of the tentacles, one end of each wire being attached to one of the slides and the other end of each wire being attached to the other slide whereby rotation in one direction of a knob attached to the screw causes the tentacles to be deflected away from the catheter shaft, and rotation of the knob in the opposite direction causes the tentacles to be retracted toward the catheter shaft.

10. An electrode catheter as recited in claim 9 wherein a tip electrode is affixed to the distal end of the distal shaft component.

11. An electrode catheter as recited in claim 10 wherein at least one ring electrode is disposed along the length of the steerable portion of the distal shaft component.

12. An electrode catheter as recited in claim 1 wherein the second means includes a user-engageable screw mechanism affixed to the handle and connected to the distal ends of the tentacles by wires extending along the length of the catheter shaft.

13. An electrode catheter as recited in claim 12 wherein the screw mechanism includes a threaded screw that engages first and second slides driven in opposite directions by the screw, and wires extending through the catheter shaft and tentacles to the distal ends of the tentacles, one end of each wire being attached to one of the slides and the other end of each wire being attached to the other slide whereby rotation in one direction of a knob attached to the screw causes the tentacles to be deflected away from the catheter shaft, and rotation of the knob in the opposite direction causes the tentacles to be retracted toward the catheter shaft.

14. An electrode catheter as recited in claim 13 wherein the first means includes a user-engageable steering lever affixed to the handle and connected to the distal end of the distal shaft component by wires extending along the length of the catheter shaft.

15. An electrode catheter as recited in claim 1 wherein the proximal end of the steerable portion of the distal shaft component is attached to the second end of the proximal shaft component, and the distal end of the non-steerable portion forms the distal end of the catheter.

16. An electrode catheter as recited in claim 15 wherein the tentacles are attached to the distal end of the steerable portion of the distal shaft component and extend over the non-steerable portion.

17. An electrode catheter as recited in claim 15 wherein the non-steerable portion of the distal shaft component provides a centralizing and stabilizing function for the tentacles.

18. An electrode catheter as recited in claim 16 wherein the first means includes a user-engageable steering lever affixed to the handle and connected to the distal end of the distal shaft component by wires extending along the length of the catheter shaft.

19. An electrode catheter as recited in claim 16 wherein the second means includes a user-engageable screw mechanism affixed to the handle and connected to the distal ends of the tentacles by wires extending along the length of the catheter shaft.

20. An electrode catheter as recited in claim 19 wherein the screw mechanism includes a threaded screw that engages first and second slides driven in opposite directions by the screw, and wires extending through the catheter shaft and tentacles to the distal ends of the tentacles, one end of each wire being attached to one of the slides and the other end of each wire being attached to the other slide whereby rotation in one direction of a knob attached to the screw causes the tentacles to be deflected away from the catheter shaft, and rotation of the knob in the opposite direction causes the tentacles to be retracted toward the catheter shaft.
